(19) United States
(12) Patent Application Publication
(10) Pub. No.: US 2022/0331301 A1
(43) Pub. Date: Oct. 20, 2022

(54) CYSTIC FIBROSIS TRANSMEMBRANE CONDUCTANCE REGULATOR MODULATORS FOR TREATING AUTOSOMAL RESESSIVE POLYCYSTIC KIDNEY DISEASE

(71) Applicant: The Johns Hopkins University, Baltimore, MD (US)

(72) Inventor: Liudmila Cebotaru, Baltimore, MD (US)

(21) Appl. No.: 17/674,090

(22) Filed: Feb. 17, 2022

Related U.S. Application Data
(60) Provisional application No. 63/152,391, filed on Feb. 23, 2021.

(51) Int. Cl. A61K 31/443; A61P 13/12; A61K 31/352; A61K 31/47

(52) U.S. Cl. CPC A61K 31/443; A61P 13/12; A61K 31/352; A61K 31/47

(57) ABSTRACT

Cystic fibrosis transmembrane conductance regulator (CFTR) modulators rescue several processes malfunctioning in autosomal recessive polycystic kidney disease (ARPKD), including increasing FPC and CFTR protein levels, increasing CFTR at the basolateral membrane and at the cilia, reducing cAMP, reducing ER Ca2+ release in response to thapsigargin, and reducing Hsp70, Hsp90, and Aha1. CFTR modulators also correct the trafficking of CFTR. All these maneuvers, either individually or in combination, reduce cyst growth in liver and improve liver, renal, pancreatic and lung function.

This application claims priority to U.S. provisional patent application Ser. No. 63/152,391, filed Feb. 23, 2021, which is incorporated herein by reference in its entirety.

### STATEMENT OF GOVERNMENTAL INTEREST

This invention was made with government support under grant DK125272 awarded by the National Institutes of Health. The government has certain rights in the invention.

### TECHNICAL FIELD

The present disclosure relates to the use of cystic fibrosis transmembrane conductance regulator modulators to treat autosomal recessive polycystic kidney disease.

### BACKGROUND

Autosomal recessive polycystic kidney disease (ARPKD) is a debilitating autosomal recessive genetic disorder responsible for chronic end stage kidney disease in humans (ESRD). Sweeney and Avner, 2006; Zerres et al., 1998. The disorder occurs in 1 in 20,000 live births in the U.S. and more than 350,000 people per year world-wide are affected, causing a large heath care burden and newborn morbidity and mortality. The most severe disease occurs in perinates with massively enlarged echogenic kidneys and oligohydramnios. Neonates with severe disease have pulmonary hypoplasia with a mortality of approximately 30%. Roy et al., 1997. Those that survive the neonatal period face a myriad of comorbidities including systemic and portal hypertension, ESRD, fibrosis of both the liver and kidney, hepatosplenomegaly, Sweeney and Avner, 2006, and enlarged kidneys, with fusiform dilation of the collecting ducts which progresses to end stage renal disease. Hartung and Guay-Woodford, 2014.

ARPKD is caused by mutations in the PKHD1 gene encoding a large 4074 amino acid protein, fibrocystin/polyductin (FPC) with a long extracellular domain and single transmembrane domain. Onuchic et al., 2002; Harris and Rossetti, 2004. Several alternate splicing forms suggest both a anchored and secreted protein. Menezes et al., 2004. The gene is expressed predominately in the fetal and adult, liver, kidney and pancreas; the protein is present in the plasma membrane, primary cilium and cytoplasm. Onuchic et al., 2002. Studies showed that, in the absence of FPC, PC2 protein expression is downregulated and PC2 generated currents disrupted indicating both a physical and functional interaction between the two proteins. Kim et al., 2008.

Diagnostic features of ARPKD are detected by ultrasound, computed tomography (CT), or genetic testing. ARPKD is hereditary. ARPKD may lead to increased renal size with increased echogenicity, and poor corticomedullary differentiation. Severely affected fetuses may have oligohydramnios leading to pulmonary hypoplasia. The biliary abnormalities may include portal hypertension. Magnetic resonance cholangiopancreatography may detect biliary duct ectasia. Individuals often have systemic hypertension. Zerres et al., 1998.

Cystic fibrosis transmembrane conductance regulator (CFTR) is a member of the ATP binding cassette family. CFTR may function as a cAMP-dependent chloride channel. Channel activation may be mediated by cycles of regulatory domain phosphorylation, ATP-binding by the nucleotide-binding domains, and ATP hydrolysis. Mutations in the CFTR gene cause cystic fibrosis. The most frequently occurring mutation in cystic fibrosis, DeltaF508, results in impaired folding and trafficking of the encoded protein. CFTR may line the luminal membrane of ARPKD cysts or be present in the basolateral membrane or within an intracellular organelle. CFTR may contribute to cAMP-dependent fluid secretion and cyst growth in ARPKD. Modulators may be used to target CFTR. Fuller and Benos, 1992.

### SUMMARY

In some aspects, the presently disclosed subject matter provides a method for treating a cystic kidney, and/or a liver, pancreatic, and associated lung disease, the method comprising administering to a subject in need of treatment thereof a therapeutically effective amount of a cystic fibrosis transmembrane conductance regulator (CFTR) modulator.

In certain aspects, the cystic kidney disease comprises autosomal recessive polycystic kidney disease (ARPKD).

In some aspects, the method further comprises treating one or more diseases selected from lung development, pancreatic, kidney, and liver function.

In some aspects, the method further comprises administering the CFTR modulator with one or more additional therapeutic agents.

In certain aspects, the CFTR modulator comprises a small molecule.

In certain aspects, the CFTR modulator comprises a potentiator. In particular aspects, the potentiator activates a channel.

In certain aspects, the CFTR modulator comprises a corrector. In particular aspects, the corrector affects protein folding.

In certain aspects, the CFTR modulator comprises an amplifier. In particular aspects, the amplifier increases gene expression.

In certain aspects, the CFTR modulator alters protein trafficking.

In certain aspects, the CFTR modulator inhibits ER Ca2+ release. In particular aspects, the inhibition of ER Ca2+ release prevents ARPKD cysts from responding to growth stimuli.

In certain aspects, the CFTR modulator changes one or more of Hsp27, Hsp90, Aha1 and/or Hsp70. In particular aspects, the changes in Hsp27, Hsp90, Aha1 and/or Hsp70 decreases a size or number of ARPKD cysts in the subject. In other aspects, the changes in Hsp27, Hsp90, Aha1 and/or Hsp70 mitigates one or more of lung hypoplasia and/or liver and pancreatic, kidney disease.

In certain aspects, the CFTR modulator decreases cAMP.

In certain aspects, the CFTR modulator increases protein expression and trafficking in one or more of the kidney, lung, pancreas, and cholangiocytes. In particular aspects, the increase in protein expression increases chloride transport.

In certain aspects CFTR modulator alters trafficking of key proteins including SNARE and PDZ domain proteins restoring their proper functioning location.

In certain aspects, the CFTR modulator prevents excessive secretion of chloride and sodium into the cyst lumen. In particular aspects, the prevention of the excessive secretion of chloride into the cyst lumen prevents sodium and water from entering the cyst lumen. In more particular aspects, preventing water from entering the cyst lumen reduces the size of a cyst. In yet more particular aspects, preventing water from entering the lumen on the cyst treats ARPKD.

In certain aspects, the CFTR modulator restores renal, pancreatic, lung and liver cells in ARPKD to a non-cyst forming phenotype. In particular aspects, the method further comprises negating the ability of cAMP to sustain and stimulate cyst growth.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1A and FIG. 1B show the expression of fibrocystin (FPC) in normal and reduced levels in Pkhd1^{del4/del4} cholangiocytes. The residual level of FPC is expected in Pkhd1^{del4/del4} cholangiocytes. Garcia-Gonzalez et al., 2007. VX-809 increases FPC. Columns represent the means±SEM of the FPC expression. The data were analyzed by non-parametric t-test. The experiment was repeated 3-4 times; P<0.05;

FIG. 2A and FIG. 2B show that CK7, a marker of cholangiocytes, Paku et al., 2005, is present in WT and Pkhd1^{del4/del4} cells verifying that these are indeed cholangiocytes;

FIG. 2C and FIG. 2D show that CK7 is reduced in Pkhd1^{del4/del4} cells and increased by VX-809;

FIG. 3A, FIG. 3B, FIG. 3C and FIG. 3D show that VX-809 reduces cysts in Pkhd1^{del4/del4} cholangiocytes. FIG. 3A shows representative images of WT and Pkhd1^{del4/del4} cholangiocytes grown on matrigel and cyst growth was monitored for 72 hrs. Note that the cysts treated with forskolin (for) grew larger. Note that VX-809 treatment reduced cyst growth in the presence of forskolin. FIG. 3B shows summary data from 3-4 experiments. FIG. 3C shows representative images of WT and Pkhd1^{del4/del4} cholangiocytes grown on matrigel and cyst growth was monitored for 1 week. Note that the cysts treated with forskolin (for) grew larger. Note that VX-809 treatment reduced cyst growth in the presence of forskolin. FIG. 3C for 1 wk. FIG. 3D shows summary data from 3-4 experiments. Significant difference in the cyst area was observed in cysts continuously treated with VX-809 (10 μM). Cysts were induced with Forskolin (10 μM) for 24 hours prior to study. Columns represent means±standard error (SEM). **P<0.01 ****P<0.0001 (for all graphs). Statistical analysis was performed using an unpaired two-tailed Student's t-test;

FIG. 4A, FIG. 4B, FIG. 4C and FIG. 4D show that FDL reduces cysts in Pkhd1^{del4/del4} cholangiocytes. FIG. 4A shows representative images of WT and Pkhd1^{del4/del4} cholangiocytes grown on matrigel and cyst growth was monitored for 48 hrs. Note that the cysts treated with forskolin (for) grew larger. Note that FDL 169 treatment reduced cyst growth in the presence of forskolin. FIG. 4B shows summary data from 3-4 experiments. FIG. 4C shows representative images of WT and Pkhd1^{del4/del4} cholangiocytes grown on matrigel and cyst growth was monitored for 1 week. Note that the cysts treated with forskolin (for) grew larger. Note that FDL 169 treatment reduced cyst growth in the presence of forskolin. FIG. 4C for 1 wk. FIG. 4D shows summary data from 3-4 experiments. Significant difference in the cyst area was observed in cysts continuously treated with FDL 169 (10 μM). Cysts were induced with Forskolin (10 μM) for 24 hours prior to study. Columns represent means±standard error (SEM). **P<0.01 ****P<0.0001 (for all graphs). Statistical analysis was performed using an unpaired two-tailed Student's t-test;

FIG. 5 shows that cAMP levels are reduced by VX-809. (FIG. 5 cAMP levels in Pkhd1^{del4/del4} cholangiocytes were reduced by VX-809. Confluent WT and Pkhd1^{del4/del4} cholangiocytes were treated with VX-809 (10 μM) 16 h and then treated with forskolin (100 μM) for 30 min before the cells were harvested for the assay. Columns represent means±SEM. Statistical analysis was performed using a two-tailed Student's t-test. Each set of data is from three individual wells; Note that resting cAMP is greater in Pkhd1^{del4/del4} cholangiocytes vs WT as previously shown. Yanda et al., 2018. Also note that forskolin caused a large increase in cAMP. VX-809 treatment reduced the levels of cAMP when compared to untreated cells, in either the presence or absence of forskolin. The experiment was repeated four times. For all graphs, P<0.05, ***P<0.001 and ****P<0.0001;

FIG. 6A, FIG. 6B, and FIG. 6C show intracellular Ca2+ (F340/F380) levels obtained by ratiometric Fura-2 AM analysis of WT and Pkhd1^{del4/del4} cholangiocytes, the latter treated with VX-809 (10 μM) for 16 h. (FIG. 6A) Representative traces of ER Ca2+ release in response to thapsigargin (4 μM) in WT and Pkhd1^{del4/del4} cholangiocytes the latter treated with VX-809. Resting calcium levels (FIG. 6B and the average amplitude of Ca2+ release (FIG. 6C) in response to thapsigargin;

FIG. 6D, FIG. 6E, and FIG. 6F show the effect of the mitochondrial Ca2+ uniporter inhibitor RU360. FIG. 6E and FIG. 6F show the effects on resting Ca2+ and the spike amplitude respectively Amplitude was measured as the standard deviation of the signal base to peak Δf/f. Significance between the two groups was analyzed using Student's t-test, n=4-5). *P;

FIG. 7A shows a western blot of CFTR protein expression in Pkhd1del4/del4 cholangiocytes vs. WT;

FIG. 7B shows the average±SE of 3-4 samples. Please note the reduction of CFTR protein in Pkhd1del4/del4 cholangiocytes and the increase following VX-809 treatment;

FIG. 7C shows a western blot of PC2 protein expression in Pkhd1del4/del4 cholangiocytes vs. WT;

FIG. 7D shows the average±SE of 3-4 samples. Please note the reduction of PC2 protein in Pkhd1del4/del4 cholangiocytes and the increase following VX-809 treatment;

FIG. 8A shows a micrograph of CFTR (red) and WGA (green) in bile duct epithelial cells of Pkhd1 Pkhd1del4/del4 cholangiocytes treated with VX-809 (10 μM). WGA is an apical membrane marker;

FIG. 8B summarizes the Pearson's Correlation coefficient R. Columns represent the means±SEM. The data shows that VX-809 reduces colocalization in Pkhd1del4/del4 cholangiocytes with the apical cell membrane;

FIG. 8C shows CFTR (red) and Na+/K+ ATPase (green) in bile duct epithelial cells of Pkhd1del4/del4 cholangiocytes treated with VX-809. The Na+/K+ ATPase is a marker of the basolateral membrane, Yanda et al., 2019a;

FIG. 8D shows a summary of Pearson's Correlation coefficient R in mutant cells. Please note that VX-809 treatment increases basolateral colocalization of CFTR in mutant cells. Confocal images taken of cells grown on transwell membranes. All images were captured with a 63× oil objective. Strips represent Z-stacks of confocal images acquired at 0.4 μm z-axis interval. Images from left to right in each row are wild-type and PKHD del4/del4 mutant cells. Scale bar is 10 μm. Unpaired t test. Western blot=Antibody 217 and Confocal=596 from the CFF antibody core at UNC;

FIG. 9A, FIG. 9B, FIG. 9C, FIG. 9D and FIG. 9E show chaperone expression is altered Pkhd1del4/del4 cholangiocytes. (FIG. 9A) Representative Western blot images of HSP27, 70, 90 and Aha1 in WT and Pkhd1del4/del4 cholangiocytes treated with VX-809 as described in previous figures. (FIG. 9B, FIG. 9C, FIG. 9D and FIG. 9E) Columns represent averages±standard errors of Hsp 27, Aha1, HSP70 and HSP90 expression. Data were analyzed by non-parametric t-test. n=4 for each treated and control groups. **P. Please note the large decrease in Hsp27 and increases in Aha1, HSP70 and Hsp 90 in Pkhd1del4/del4 cholangiocytes compared to WT. Also, there is a correction toward WT values after Pkhd1del4/del4 cholangiocytes are treated with VX-809;

FIG. 10A and FIG. 10B show that cyst area is reduced by transfection of Hsp27.

FIG. 10C and FIG. 10D show that cyst area is reduced by a Hsp90 inhibitor; and

FIG. 10E and FIG. 10F show cyst area is reduced by silencing Hsp90.

FIG. 11A and FIG. 11B show images of livers from WT, untreated Pkhd1^{del3-4/del3-4} mice, and Pkhd1^{del3-4/del3-4} mice treated with VX-809 for one month starting at five days old. FIG. 1A shows 4× light microscope images of H & E stained livers from WT, Pkhd1^{del3-4/del3-4} and Pkhd1^{del3-4/del3-4} mice treated with VX-809. Black arrowheads indicate the bile ducts and the green arrowheads indicate the portal veins. Scalebar is 100 μm. FIG. 11B shows confocal immunofluorescence 63× images of CK19 expression, a cholangiocyte marker of bile duct epithelium (see, e.g., Tabibian (2013) Physiology of cholangiocytes. Compr Physiol 3, 541-565, incorporated herein by reference) in WT, Pkhd1^{del3-4/del3-4} and Pkhd1^{del3-4/del3-4} treated with VX-809. Scalebar is 10 μm. As indicated by the images, normal animals have small bile ducts near to the portal veins, whereas the ducts in the ARPKD animals are enlarged and malformed. VX-809 restores the bile ducts toward normal.

FIG. 12A and FIG. 12B show Ki67 expression in WT and Pkhd1^{del3-4/del3-4} mice bile duct epithelia. FIG. 12A shows confocal laser-scanning immunofluorescence microscopy images of mice liver paraffin sections taken from WT (NoDoxy Pkd1), untreated Pkhd1^{del3-4/del3-4} mice, and Pkhd1^{del3-4/del3-4} mice treated with VX-809 and stained for Ki67, a marker of proliferation (red), and cytokeratin 19 (CK19), a marker of cholangiocytes in the bile duct (green). Merged red and green channels are shown, wherein yellow indicates colocalization; the white square indicates the area of enlargement shown. FIG. 12B summarizes the Pearson's Correlation coefficient R for the images of FIG. 12A. The graph compares the percentage of cells positive for Ki67. Number of cells analyzed for WT, n=196, Pkhd1^{del3-4/del3-4}, n=324, Pkhd1^{del3-4/del3-4}+VX-809, n=344 Samples were incubated with 5 μg/ml primary antibody Ki67 (Abcam #ab15580) and CK19 (Invitrogen #MA5-15884) in blocking buffer for 16 hours at 4° C. Goat anti-rabbit Alexa Fluor 594 and anti-mouse Alexa Flour 488 conjugated secondary antibodies were used at a 1/250 dilution for 1 hour to label the proteins. Scale bar is 10 μm. Bars represent the means±SEM. The data show that proliferation as detected by increased Ki67 is higher in the Pkhd1^{del3-4/del3-4} mouse tissue compared to WT mouse. VX-809 reduces colocalization in Pkhd1^{del3-4/del3-4} cholangiocytes with Ki67 indicative of reduced proliferation. *P<0.05; ****P<0.0001. Percent cells positive for ki67 are significantly higher in Pkhd1^{del3-4/del3-4} compared to WT or VX-809-treated Pkhd1^{del3-4/del3-4} groups. These data indicate that proliferation is higher in Pkhd1^{del3-4/del3-4} compared to controls and that the levels were reduced following VX-809 treatment.

FIG. 13A and FIG. 13B show CFTR and CK19 colocalization in WT and Pkhd1^{del3-4/del3-4} mice bile duct epithelia. FIG. 13A shows confocal laser-scanning immunofluorescence microscopy images of mice liver paraffin sections taken from WT (NoDoxy Pkd1), untreated Pkhd1^{del3-4/del3-4} mice, and Pkhd1^{del3-4/del3-4} mice treated with VX-809 and stained for CFTR (red) and CK19 (green) in bile duct epithelial cells. Merged red and green channels are shown, wherein yellow indicates colocalization; the white square indicates the area of enlargement shown. FIG. 13B summarizes the Pearson's Correlation coefficient R. Bars represent the means±SEM. Samples were incubated with 5 μg/ml primary antibody CFTR (596) and CK19 (Invitrogen #PA5-29548) in blocking buffer for 16 hours at 4° C. Goat anti-rabbit Alexa Fluor 594 and anti-mouse Alexa Flour 488 conjugated secondary antibodies were used at a 1/250 dilution for 1 hour to label the proteins. Scale bar is 10 μm. **P<0.01, ****P<0.0001. These data show that These data show that CFTR is higher in Pkhd1^{del3-4/del3-4} cholangiocytes and that CFTR-CK19 colocalization is significantly higher in the ARPKD untreated group compared to wild type. VX-809 treatment significantly lowered the colocalization.

FIG. 14A and FIG. 14B show CFTR and E-Cadherin colocalization in WT and Pkhd1^{del3-4/del3-4} mice bile duct epithelia. FIG. 14A shows confocal laser-scanning immunofluorescence microscopy images of mice liver paraffin sections taken from WT (NoDoxy Pkd1), untreated Pkhd1^{del3-4/del3-4} mice, and Pkhd1^{del3-4/del3-4} mice treated with VX-809 and stained for CFTR (red) and E-Cadherin (green) in bile duct epithelial cells. Merged red and green channels are shown, wherein yellow indicates colocalization; the white square indicates the area of enlargement shown. E-Cadherin is a marker of the lateral junctional membrane of epithelial cells (see, e.g., Bruser (2017) Adherens Junctions on the Move-Membrane Trafficking of E-Cadherin. Cold Spring Harb. Perspect. Biol. 9, a029140, incorporated herein by reference). FIG. 14B summarizes the Pearson's Correlation coefficient R in mutant cells. Samples were incubated with 5 μg/ml primary antibody CFTR (596) and eCadherin (R&D #AF748) in blocking buffer for 16 hours at 4° C. Goat anti-mouse Alexa Fluor 594 and anti-goat Alexa Flour 488 conjugated secondary antibodies were used at a 1/250 dilution for 1 hour to label the proteins. Scale bar is 10 μm. As shown by the data, Pkhd1^{del3-4/del3-4} cholangiocytes have much greater colocalization of CFTR with the apical/lateral marker compared to WT cells. VX-809 treatment decreases E-cadherin colocalization of CFTR in mutant mice cells. All images were captured with a 63× oil objective. Significance was analyzed using unpaired t test. Confocal=596 from the CFF antibody core at UNC. These data show that CFTR-eCadherin colocalization is significantly higher in ARPKD untreated group compared to wild type. VX-809 treatment significantly lowered the colocalization.

FIG. 15A and FIG. 15B show CFTR and Na/K ATPase colocalization in WT and Pkhd1^{del3-4/del3-4} mice bile duct epithelia. FIG. 15A shows confocal laser-scanning immunofluorescence microscopy images of mice liver paraffin sections taken from WT (NoDoxy Pkd1), untreated Pkhd1^{del3-4/del3-4} mice, and Pkhd1^{del3-4/del3-4} mice treated with VX-809 and stained for CFTR (red) and Na⁺/K⁺ ATPase (green) in bile duct epithelial cells. Merged red and green channels are shown, wherein yellow indicates colocalization; the white square indicates the area of enlargement shown. The Na⁺/K⁺ ATPase is a marker of the basolateral membrane (see, e.g., Burgoyne (2015) Calcium signaling at ER membrane contact sites. Biochim. Biophys. Acta 1853, 2012-2017, incorporated herein by reference). FIG. 15B summarizes Pearson's Correlation coefficient R in mutant mice cells. Samples were incubated with 5 μg/ml primary antibody CFTR (596) and Na/K ATPase (Abcam #ab76020) in blocking buffer for 16 hours at 4° C. Goat anti-rabbit Alexa Fluor 594 and anti-mouse Alexa Flour 488 conjugated secondary antibodies were used at a 1/250 dilution for 1 hour to label the proteins. Scale bar is 10 μm. *P<0.05. Confocal images taken from sections of liver tissue from the mice. All images were captured with a 63× oil objective. Significance was analyzed using unpaired t test. Confocal=596 from the CFF antibody core at UNC. These data show that CFTR-Na/K ATPase colocalization is significantly higher in the WT group compared to the untreated control group and that VX-809 treatment significantly increased CFTR-Na/K ATPase colocalization in mutant mice cells compared to untreated group.

FIG. 16A, FIG. 16B, and FIG. 16C show Ca²⁺ release in a renal ARPKD cell line treated with VX-809. Intracellular Ca²⁺ (F340/F380) levels were measured by ratiometric Fura-2 AM analysis of WT and Pkhd1 kidney cells treated with VX-809 (10 μM) for 16 hours. FIG. 16A shows representative traces of ER Ca′ release in response to thapsigargin (4 μM) in WT and Pkhd1 kidney cells treated with VX-809. FIG. 16B shows resting Ca²⁺ levels. FIG. 3C shows average amplitude of Ca²⁺ release in response to thapsigargin. Amplitude was measured as the standard deviation of the signal base to peak Δf/f. Asterisk indicates significance between the two groups. Significance between the two groups was analyzed using Student's t-test, n=5-6. ****P<0.0001. These data show that resting Ca²⁺ was not affected by treatment with VX-809 but the release of Ca²⁺ from the ER was greater in ARPKD relative to WT. VX-809 (10 μM) treatment significantly lowered the thapsigargin-induced ER Ca²⁺ compared to untreated control cells.

FIG. 17A-17F show Ca²⁺ release in PLD human liver cell lines treated with VX-809. Intracellular Ca²⁺ (F340/F380) levels were measured by ratiometric Fura-2 AM analysis of WT and PLD liver cells treated with VX-809 (10 μM) for 16 hours. FIG. 7A FIG. 7B show representative traces of ER Ca²⁺ release in response to thapsigargin (4 μM) in WT and PLD liver cells treated with VX-809. FIG. 7C and FIG. 7D show resting Ca²⁺ levels. FIG. 7E and FIG. 7F show the average amplitude of Ca²⁺ release in response to thapsigargin. Amplitude was measured as the standard deviation of the signal base to peak Δf/f. Significance between the two groups was analyzed using Student's t-test, n=5-6. ****P<0.0001. Th These data show that resting Ca²⁺ was not affected by treatment with VX-809 but the release of Ca²⁺ from the ER was greater in ARPKD relative to WT. VX-809 (10 μM) treatment significantly lowered the thapsigargin-induced ER Ca²⁺ compared to untreated control cells.

FIG. 18 shows cAMP levels in ARPKD renal cells. Human ARPKD confluent cells were treated with VX-809 (10 mM) for 16 hours before harvesting the cells for assay. Cyclic AMP levels were measured with a direct cAMP enzyme immunoassay kit (CA 200, Sigma) using the manufacturer's protocol. Results are expressed as pmol/ml (each bar shows the results from 5-6 individual dishes). Resting cAMP is greater in ARPKD relative to WT and was reduced by VX-809. Bars represent averages±SEM. Statistical analysis was performed using unpaired t test n=7-8. ***P<0.001, ****P<0.0001. These data show that cAMP levels were higher in ADPKD cells compared to normal cells and that VX-809 treatment reduced the cAMP levels to normal.

FIG. 19 shows cAMP levels in PLD liver cell lines. Cyclic AMP levels were measured with a direct cAMP Enzyme immunoassay kit (CA 200, Sigma) using the manufacturer's protocol. Results are expressed as pmol/ml (each bar shows the results from 6-8 individual dishes). Bars represent averages±SEM. Statistical analysis was performed using unpaired t test n=7-8, *P<0.05, **P<0.01, ***P<0.001. cAMP levels were higher in PLD cells compared to normal cells and VX-809 treatment lowered the cAMP levels compared to normal controls.

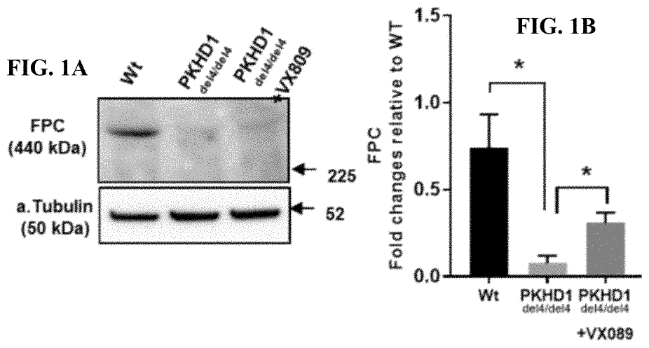

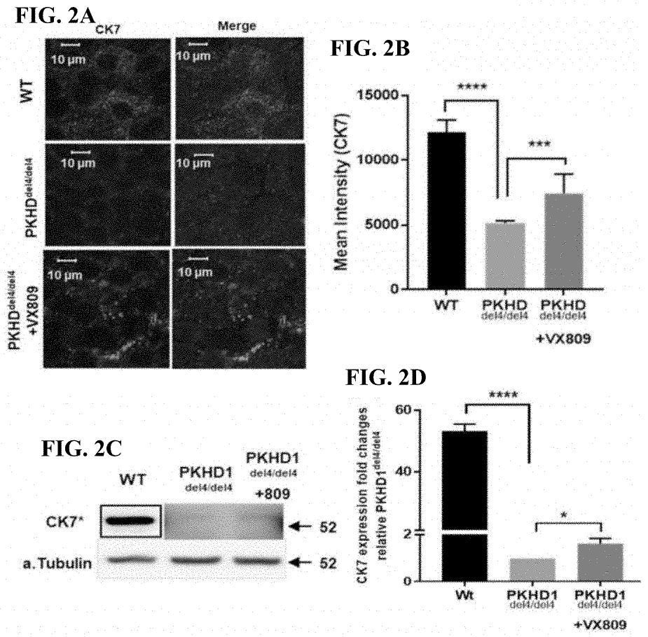

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Methods for Treating Autosomal Recessive Cystic Kidney Disease

The disclosed modulators may be used in methods for treatment of autosomal recessive cystic kidney disease. The methods of treatment may comprise administering to a subject in need of such treatment a composition comprising a therapeutically effective amount of the modulators disclosed herein. Treatment of such autosomal recessive cystic kidney diseases affect, in addition to kidney disease, lung development, pancreatic and liver function.

Modulators of this disclosure may be administered alone or in combination with another active agent as part of a therapeutic regimen to a subject in need thereof. Specifically, the methods of treatment disclosed herein may treat autosomal recessive polycystic kidney disease.

Modulators

The CFTR modulator may be a small molecule. A modulator may be potentiator, which may activate a channel. A modulator may be a corrector which may affect protein folding. A modulator may be an amplifier which may increase gene expression. A modulator may alter protein trafficking. The modulator may inhibit ER Ca2+ release. The inhibition of ER Ca2+ release may prevent ARPKD cysts from responding to growth stimuli. The modulator may change Hsp27, Hsp90, Aha1 and/or Hsp70. Induced changes in Hsp27, Hsp90, Aha1 and/or Hsp70 may decrease the size or number of ARPKD cysts and mitigate other symptoms, such as lung hypoplasia and liver disease. The modulator may decrease cAMP. The modulator may increase CFTR protein expression and/or processing and trafficking in the kidney, lung, pancreas and cholangiocytes. Increased CFTR protein expression may lead to an increase in chloride transport. In certain embodiments, the CFTR modulator alters trafficking of key proteins including SNARE and PDZ domain proteins restoring their proper functioning location. The modulator may prevent the excessive secretion of chloride and sodium into the cyst lumen. The prevention of the excessive secretion of chloride into the cyst lumen may prevent sodium and water from entering the cyst lumen. Preventing water from entering the cyst lumen may reduce the size of a cyst. Preventing water from entering the lumen on the cyst may treat ARPKD. The modulator may restore renal cells in ARPKD to a non-cyst forming phenotype, including negating the ability of cAMP to sustain and stimulate cyst growth. Modulators may directly act on CFTR to attenuate the deleterious effects of disease.

In one embodiment, disclosed are cystic fibrosis transmembrane conductance regulator (CFTR) modulators. A modulator may be potentiator. A potentiator may activate a channel. Representative potentiators include, but are not limited to, ivacaftor (VX-770).

A modulator may be a corrector. A corrector may affect protein folding.

A modulator may be an amplifier. An amplifier may increase gene expression. Generally, a CFTR amplifier enhances the effect of a potentiator or corrector. Examples of CFTR amplifiers are PTI 130 and PTI-428. Examples of amplifiers also are disclosed in WO2015138909 and WO2015138934, each of which is incorporated by reference in its entirety.

The presently disclosed methods also can include a CFTR stabilizer. A CFTR stabilizer can enhance the stability of corrected CFTR that has been treated with a corrector, corrector/potentiator, or CFTR modulator combinations. An example of a CFTR stabilizer is cavosonstat (N91115). Examples of stabilizers are also disclosed in WO2012048181, which is incorporated by reference in its entirety.

In some embodiments of the presently disclosed methods, the CFTR modulator is selected from the group consisting of a potentiator, a corrector, an amplifier, and combinations thereof. In particular embodiments, the CFTR modulator can be a corrector. In other embodiments, the CFTR modulator can be a potentiator. In other embodiments, the CFTR modulator can be an amplifier. In some embodiments, the CFTR modulator can include a combination of a corrector and a potentiator; a combination of a corrector and an amplifier; or a combination of a corrector, a potentiator, and an amplifier. In yet other embodiments, the presently disclosed methods can include a stabilizer in combination with a CFTR modulator, such as a potentiator, a corrector, and/or an amplifier.

Further, a modulator may alter protein trafficking. The modulator may reduce ER Ca²⁺ release. The modulator may inhibit ER Ca²⁺ release. The inhibition of ER Ca²⁺ release may prevent ADPKD cysts from responding to growth stimuli. The modulator may change Hsp27, Hsp90, Aha1, and/or Hsp70 activity. The changes in Hsp27, Hsp90, Aha1 and/or Hsp70 may decrease the size or number of ADPKD cysts. The modulator may decrease cAMP. The modulator may reduce cAMP levels by reducing AC3. The modulator may increase CFTR protein expression in the kidney. Increased CFTR protein expression in the kidney may lead to an increase in chloride. The modulator may prevent the excessive secretion of chloride into the cyst lumen. The prevention of the secretion of chloride into the cyst lumen may prevent sodium and water from entering the cyst lumen. Preventing water from entering the cyst lumen may reduce the size of a cyst. Preventing water from entering the lumen of the cyst may treat ADPKD. The modulator may restore renal, lung, liver and pancreatic cells in ADPKD to a non-cyst forming phenotype, including negating the ability of cAMP to sustain and stimulate cyst growth. The modulator may lead to sodium reabsorption. The modulator may restore sodium reabsorption. The modulator may move CFTR from the ER to Basolateral and Apical Membranes. The modulator may move PC2 from the ER to the Golgi.

The modulator may reduce cyst growth in the proximal tubule (PT) of the kidney, distal tubule (DT) of the kidney, and/or the collecting duct of the kidney. The modulator may restore AQP2 in the collecting duct. The modulator may lead to sodium, chloride and water reabsorption thereby reducing cyst size by absorbing fluid from the cyst lumen Modulators may directly act on CFTR to attenuate the deleterious effects of disease. Modulators may act indirectly on CFTR to attenuate the deleterious effects of the disease.

Examples of CFTR modulators that may be used with methods disclosed herein include, but are not limited to, lumacaftor (VX-809), Corr-4a, VRT-325, Cl8, C4, C3, VX-770, VX-786, 4-phenylbutyrate (4PBA), VRT-532, N6022, miglustat, sildenafil and analogs thereof, ataluren (PTC124), oubain, roscovitine, suberoylanilide hydroxamic acid, latonduine and analogs thereof, SAHA, FDL169, tezacaftor (VX-661), VX-659, and VX-445. Additional potentiators and correctors are included in U.S. Pat. No. 9,981,910, which is incorporated by reference in its entirety.

Modes of Administration

Methods of treatment may include any number of modes of administering a presently disclosed modulator. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire®). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally, for parenteral administration, the agent can be in the form of an aqueous, lipid, oily or other kind of solution or suspension or even administered in the form of liposomes or nano-suspensions.

Combination Therapies

The term “combination” is used in its broadest sense and means that a subject is administered at least two agents. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form In another embodiment, the active agents are administered in separate dosage forms (e.g., wherein it is desirable to vary the amount of one but not the other). The single dosage form may include additional active agents for the treatment of the disease state.

Further, the presently disclosed compositions can be administered alone or in combination with adjuvants that enhance stability of the agents, facilitate administration of pharmaceutical compositions containing them in certain embodiments, provide increased dissolution or dispersion, increase activity, provide adjuvant therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the modulators can be varied so long as the beneficial effects of the combination of these agents are achieved. Accordingly, the phrase “in combination with” refers to the administration of at least two modulators, and optionally additional agents either simultaneously, sequentially, or a combination thereof. Therefore, a subject administered a combination of at least two inhibitors, and optionally additional agents can receive at least two inhibitors, and optionally additional agents at the same time (i.e., simultaneously) or at different times (i.e., sequentially, in either order, on the same day or on different days), so long as the effect of the combination of all agents is achieved in the subject.

When administered sequentially, the agents can be administered within 1, 5, 10, 30, 60, 120, 180, 240 minutes or longer of one another. In other embodiments, agents administered sequentially, can be administered within 1, 2, 3, 4, 5, 10, 15, 20 or more days of one another. Where the agents are administered simultaneously, they can be administered to the subject as separate pharmaceutical compositions, each comprising either at least one inhibitor, and optionally additional agents, or they can be administered to a subject as a single pharmaceutical composition comprising all agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an agent and at least one additional therapeutic agent is greater than the sum of the biological activities of the respective agents when administered individually.

Synergy can be expressed in terms of a “Synergy Index (SI),” which generally can be determined by the method described by F. C. Kull et al. Applied Microbiology 9, 538 (1961), from the ratio determined by:

QₐQ_{A}+Q_{b}Q_{B}=Synergy Index (SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Pharmaceutical Compositions

The disclosed modulators may be incorporated into pharmaceutical compositions suitable for administration to a subject (such as a patient, which may be a human or non-human).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of modulators of the disclosure are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

For example, a therapeutically effective amount of disclosed modulators may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed modulators are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, transdermal, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound, and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type. The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid compositions, which may be administered orally, may include a disclosed immunogenic proteins, compositions, and vaccines and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed modulators may be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. The carrier of the topical composition preferably aids penetration of the compounds into the skin. The carrier may further include one or more optional components. Transdermal administration may be used to facilitate delivery.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

In an embodiment, the pharmaceutical composition may include human breast milk. The active pharmaceutical ingredient may be a component of human breast milk. The human breast milk may thus be administered to a subject in need of the active pharmaceutical ingredient.

Kits

The modulators may be included in kits comprising the immunogenic proteins, compositions, and information, instructions, or both that use of the kit will provide treatment for medical conditions in mammals (particularly humans). The kit may include an additional pharmaceutical composition for use in combination therapy. The kit may include buffers, reagents, or other components to facilitate the mode of administration. The kit may include materials to facilitate nasal mucosal administration. The kit may include materials that facilitate sublingual administration. The information and instructions may be in the form of words, pictures, or both, and the like. In addition or in the alternative, the kit may include the medicament, a composition, or both; and information, instructions, or both, regarding methods of application of medicament, or of composition, preferably with the benefit of treating or preventing medical conditions in mammals (e.g., humans). The modulators of the invention will be better understood by reference to the following examples, which are intended as an illustration of and not a limitation upon the scope of the invention.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The terms “administration” or “administering” as used herein may include the process in which the modulator as described herein, alone or in combination with other compounds or compositions, are delivered to a subject. The modulator may be administered in various routes including, but not limited to, oral, mucosal, mucosal nasal, parenteral (including intravenous, intra-arterial, and other appropriate parenteral routes), intrathecally, intramuscularly, subcutaneously, colonically, rectally, and nasally, transcutaneously, among others. The dosing of the modulator described herein to obtain a therapeutic or prophylactic effect may be determined by the circumstances of the subject, as known in the art. The dosing of a subject herein may be accomplished through individual or unit doses of the modulator herein or by a combined or prepackaged or pre-formulated dose of the modulator.

Administration may depend upon the amount of modulator administered, the number of doses, and duration of treatment. For example, multiple doses of the modulator may be administered. The frequency of administration of the immunogenic proteins, compositions, and vaccines may vary depending on any of a variety of factors. The duration of administration of the modulator, e.g., the period of time over which the modulator is administered, may vary, depending on any of a variety of factors, including subject response, etc.

The amount of the modulator administered may vary according to factors such as the degree of susceptibility of the individual, the age, sex, and weight of the individual, idiosyncratic responses of the individual, the dosimetry, and the like. Detectably effective amounts of the immunogenic proteins, compositions, and vaccines of the present disclosure may also vary.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “parenterally,” as used herein, refers to modes of administration which include intravenous, intramuscular, intraperitoneal, intrasternal, subcutaneous and intraarticular injection and infusion.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and includes an excipient, diluent, carrier, and adjuvant that are acceptable for veterinary use and/or human pharmaceutical use. “A pharmaceutically acceptable excipient, diluent, carrier and/or adjuvant” as used herein includes one or more such excipients, diluents, carriers, and adjuvants.

As used herein, the term “subject,” “patient,” or “organism” includes humans and mammals (e.g., mice, rats, pigs, cats, dogs, and horses). Typical subjects to which an agent(s) of the present disclosure may be administered may include mammals, particularly primates, especially humans. For veterinary applications, suitable subjects may include, for example, livestock such as cattle, sheep, goats, cows, swine, and the like; poultry such as chickens, ducks, geese, turkeys, and the like; and domesticated animals particularly pets such as dogs and cats. For diagnostic or research applications, suitable subjects may include mammals, such as rodents (e.g., mice, rats, hamsters), rabbits, primates, and swine such as inbred pigs and the like. The subject may have cystic kidney disease. The subject may have autosomal dominant polycystic kidney disease. The subject may be at risk for developing a cystic kidney disease.

The “therapeutically effective amount” for purposes herein may be determined by such considerations as are known in the art. A therapeutically effective amount of a compound may include the amount necessary to provide a therapeutically effective result in vivo. The amount of the compound or composition must be effective to achieve a response, including but not limited to a total prevention of (e.g., protection against) of a condition, improved survival rate or more rapid recovery, improvement or elimination of symptoms associated with the condition (such as cancer), or other indicators as are selected as appropriate measures by those skilled in the art. As used herein, a suitable single dose size includes a dose that is capable of preventing or alleviating (reducing or eliminating) a symptom in a subject when administered one or more times over a suitable time period. The “therapeutically effective amount” of a compound or composition as described herein may depend on the route of administration, type of subject being treated, and the physical characteristics of the subject. These factors and their relationship to dose are well known to one of skill in the medicinal art, unless otherwise indicated.

As used herein, “treat”, “treatment”, “treating”, and the like refer to acting upon a condition with an agent to affect the condition by improving or altering it. The condition includes, but is not limited to cystic kidney disease. The cystic kidney disease may be autosomal dominant polycystic kidney disease. The aforementioned terms cover one or more treatments of a condition in a subject (e.g., a mammal, typically a human or non-human animal of veterinary interest), and include: (a) reducing the risk of occurrence of the condition in a subject determined to be predisposed to the condition but not yet diagnosed, (b) impeding the development of the condition, and/or (c) relieving the condition, e.g., causing regression of the condition and/or relieving one or more condition symptoms (e.g., treating cystic kidney disease, reducing the size and/or number of cysts).

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### VX-809 Reduces Cyst Growth in Pkhd1del4/del4 Cholangiocytes

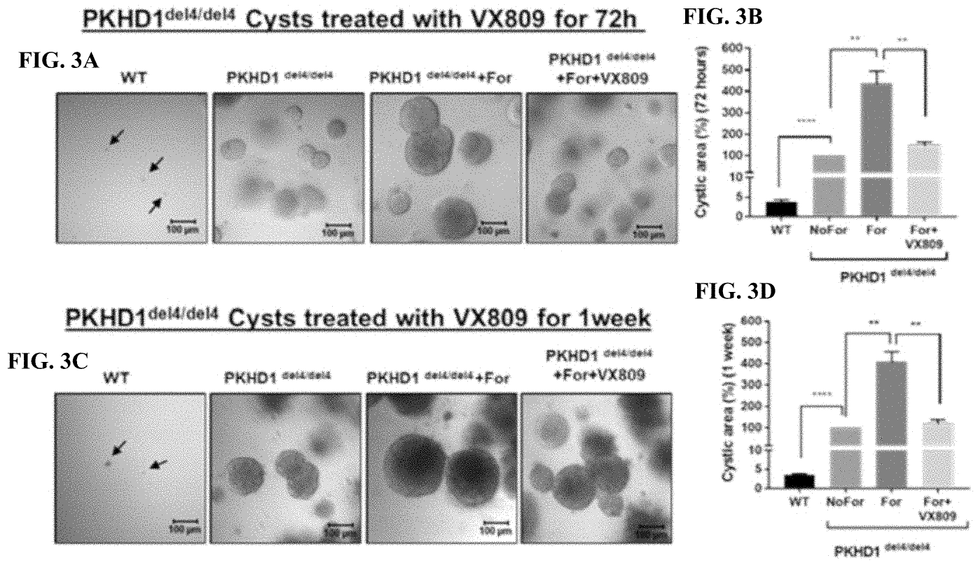

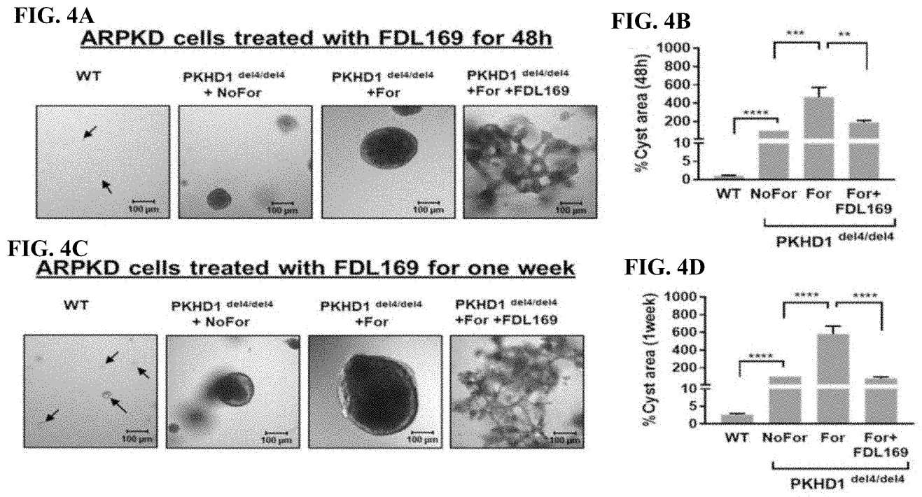

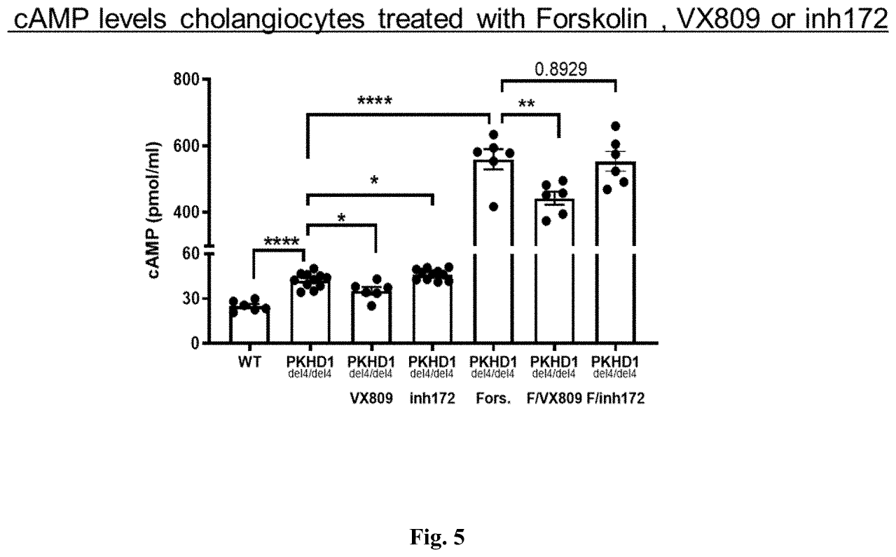

To demonstrate that CFTR correctors can affect the growth of cholangiocytes, we grew normal and Pkhd1del4/del4 cholangiocytes in 3D culture. FIG. 3 and FIG. 4 show that no cysts develop in 3D culture from wt cells, but will grow from the mutant cells. But cysts do develop from the Pkhd1del4/del4 cholangiocytes. Cyst growth is accelerated by forskolin (for). Please note that the CFTR correctors VX-809 and FL169 reduces growth. We have shown previously that VX-809 can reduce cysts in renal cell models of ADPKD and in PC1 null mice. Yanda et al., 2018. The dosage of VX-809 injected was lower than the human pediatric dose, which is 500 mg of Lumacaftor per day; the adult dose is 800 mg/day.

### Example 2

### VX-809 Downregulates cAMP Levels

Because cyst growth is accelerated by forskolin, we measured cAMP levels in these cells. FIG. 5 shows that Pkhd1del4/del4 cholangiocytes have higher cAMP levels that wt cells. VX-809 reduces cAMP levels in Pkhd1del4/del4 cholangiocytes both in the presence and absence of cAMP. As shown in FIG. 3 and FIG. 4, VX-809 can reduce cyst growth even in the presence of forskolin, which increases intracellular cAMP dramatically, points to a mechanism of action of VX-809 outside of its ability to reduce cAMP. Please note that VX-809 the corrector, reduces cAMP more effectively than the inhibitor 172.

### Example 3

### VX-809 Downregulates Release of Ca2+ from the ER

The ER is one of the major repositories for Ca2+ in the cell. Burgoyne et al., 2015. Misregulation of Ca2+ signaling is a hallmark of ADPKD. Yanda et al., 2019b. To understand whether the ER Ca2+ movement is altered, we measured ER Ca2+ release; we treated the cells with thapsigargin, a specific inhibitor of the ER Ca2+-ATPase that, when applied, allows Ca2+ to leak out of the ER through independent Ca2+-permeable pathways. Jackson et al., 1988; Thastrup et al., 1990; Thastrup et al., 1989.

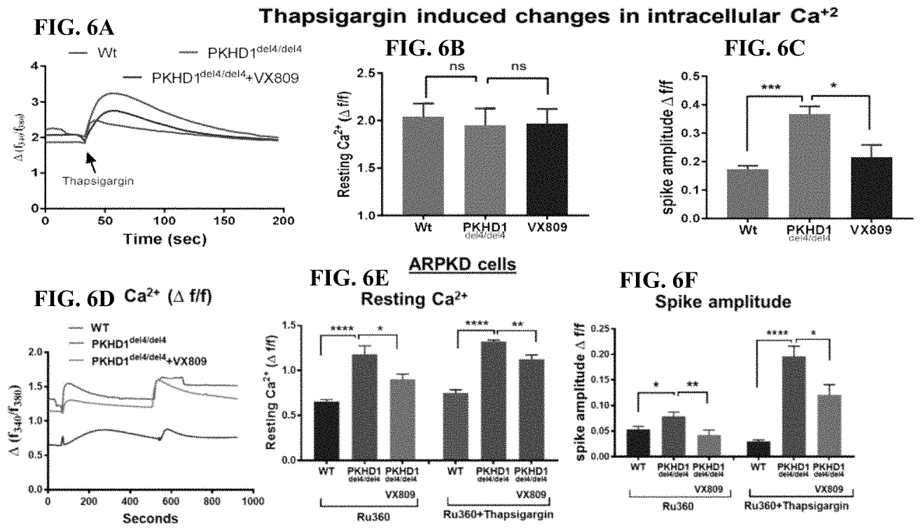

The first observation of interest is that the magnitude of the thapsigargin-induced increase in intracellular Ca2+ was >3-fold greater in Pkhd1del4/del4 cholangiocytes (FIG. 6A, FIG. 6B). This result is consistent with our previous data showing that Ca2+ signaling is elevated in PC1-knockout cells. Li et al., 2009. Importantly, VX-809 dramatically reduced the thapsigargin-induced ER Ca2+ release, to values near to those observed in wt cells.

### Example 4

### Loss of FPC Affects the Steady State Levels of CFTR and its Localization

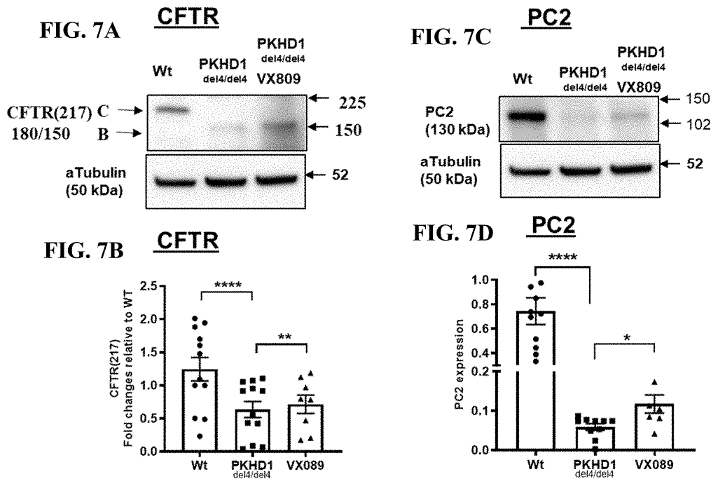

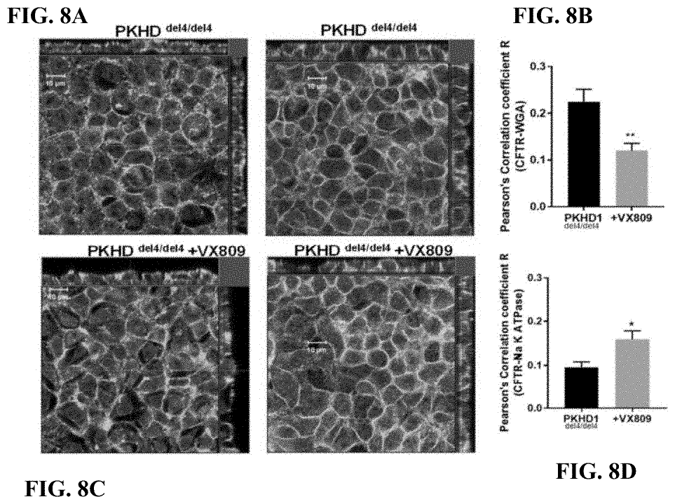

Loss of FPC causes a reduction in CFTR and PC2 levels which trends back to normal after treatment with VX-809 (FIG. 7). Loss of FPC favors the localization of CFTR at the apical cell membrane (FIG. 8), whereas treatment with VX-809 increases the colocalization with the basolateral membrane. These changes in CFTR are likely to contribute significantly to the pathophysiology of ARPKD and return toward normal important therapeutically.

### Example 5

### FPC Malfunction Alters the Steady-State Levels of Heat Shock Proteins

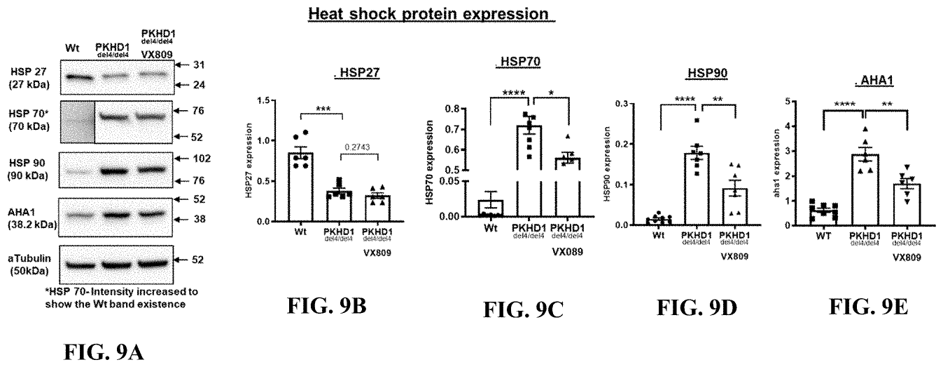

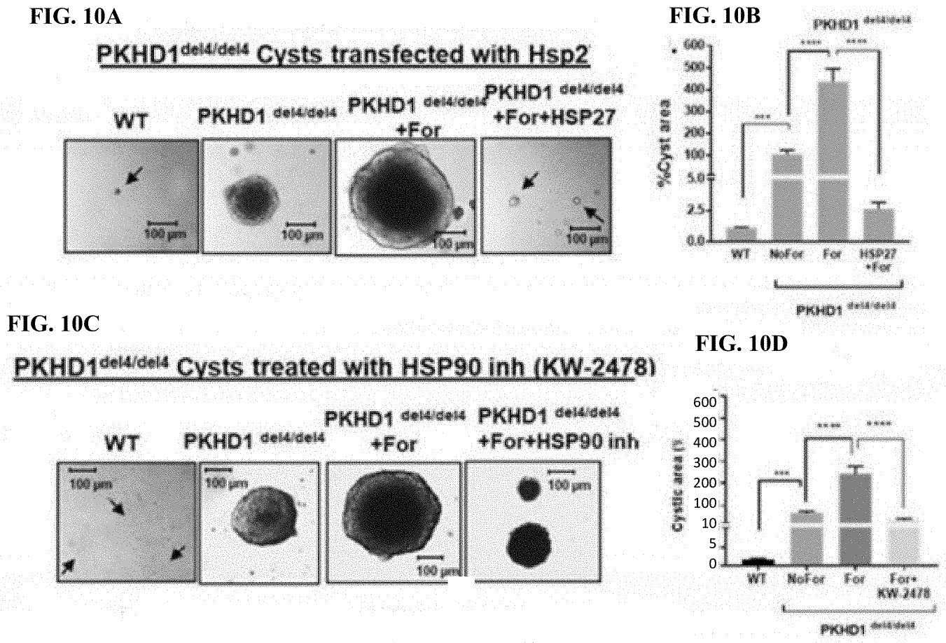

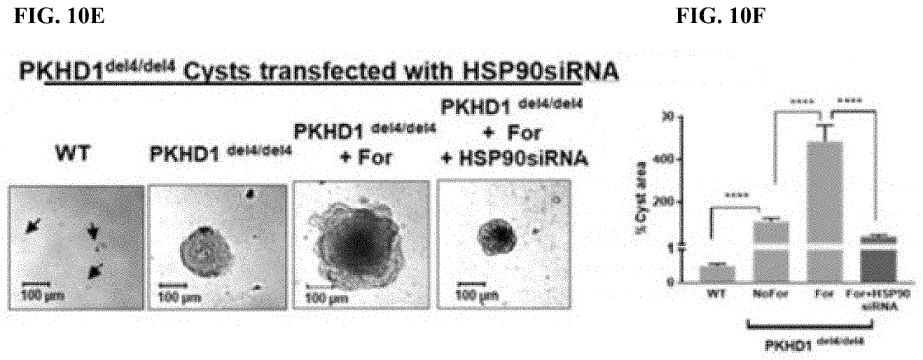

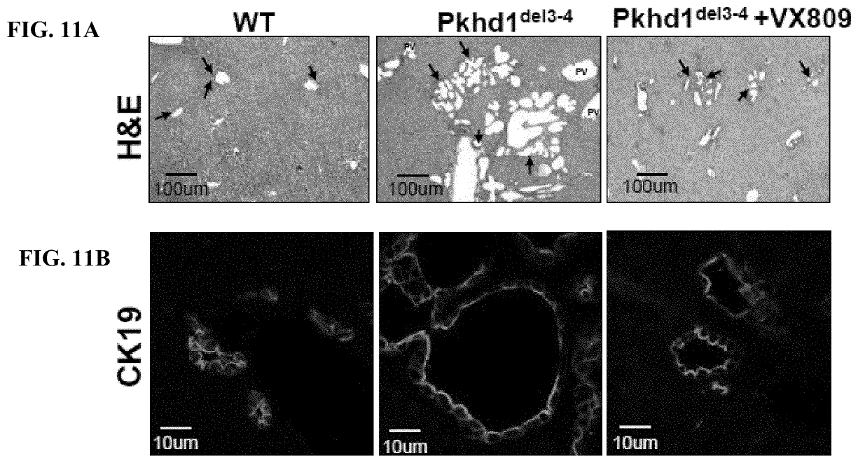

We have previously shown that CFTR-based correctors rescue ΔF508-CFTR by altering the proteostatic network of ER quality control proteins (HSPs) involved in its degradation. Lopes-Pacheco et al., 2015. It is known that HSP90 is hyperactive in ADPKD cysts and that inhibiting its function with STA-2842, Seeger-Nukpezah et al., 2013, reduces cyst growth. To continue this line of investigation, we measured the steady-state levels of HSPs in Pkhd1del4/del4 cholangiocytes (FIG. 9). In the mutant cholangiocytes, HSP27 was reduced by half whereas, HSP70 was increased by 8 fold, HSP90 was by 2-fold and Aha1 by 2.5 fold. Treating Pkhd1del4/del4 cholangiocytes with VX-809 reduced the levels of HSP 70, 90 and Aha1 but did not change the level of Hsp27.

### Example 6

### Cyst Growth is Reduced by Altering Hsps

To delineate the role of heat shock protein we transfected additional Hsp27 into Pkhd1del4/del4 cholangiocytes. Please note that cysts did not develop in the cholangiocytes transfected with additional Hsp27 indicating strongly that Hsp27 plays a role in this process. We reduced Hsp90 by mRNAi and by the inhibitor 2478. Both maneuvers reduce cyst growth. Alterations in Hsps likely plays a role in the pathophysiology of ARPKD and returning them toward normal will be important therapeutically.

In summary, VX-809 rescues several processes malfunctioning in ARPKD. VX-809 increases FPC and CFTR protein levels, increases CFTR at the basolateral membrane and at the cilia, reduces cAMP, reduces ER Ca2+ release in response to thapsigargin, and reduces Hsp 70, Hsp90, and Aha1. All these maneuvers either individually or in combination reduce cyst growth in liver and would improve liver, renal and lung function.

### Example 7

### Experimental Methods

7.1 Isolation of Cholangiocytes from Pkhd1del4/del4: Large T Mice

(Methods and cells provided by the Yale O'Brian Center) To create the cholangiocyte cultures PKhd1del/4/del4, Garcia-Gonzalez et al., 2007, animals were crossed with mice transgenic for the Sv40 large T Antigen (strain name SV40 T-antigen JAX strain #002233). Segments of intrahepatic bile ducts were microdissected from livers perfused via the portal vein with molten agarose containing toluidine blue to mark the veins. The liver parenchyma was stripped until the biliary tree was identified and the bile duct units were seeded onto rat type I collagen coated plates. Cells were cultured under non-permissive conditions in the following medium: DMEM/F12 with 5% fetal bovine serum, 10 μL/mL insulin-transferrin selenium, 3.4 μg/mL triiodothyronine, 50,000 U penicillin/streptomycin under humidified conditions at 37° C. in 5% CO₂. After one week, the cells were cultured under permissive conditions in the presence of 10 U of interferon-γ at 33° C. in 5% CO₂. When confluent, cells were passaged by digesting the collagen slab below the cells (60 min; 37° C.; DMEM/F12 with 2 mg/mL dispase and 1 mg/mL collagenase type 11), then washed twice in PBS and plated onto collagen coated support systems. Clonal cell lines were generated by single cell isolation. Cells were differentiated for 10 days at 37° C. without interferon-γ for experimental use.

7.2 Reagents

Forskolin (#11018) was purchased from Sigma (SC23950); VX-809 (#S1565) was purchased from Selleck chemicals, Huston, Tex., USA; Ezrin (SC58758) adenylate cyclase 3 (SC588), PC2 (SC28331), Hsp27 (SC13132), Hsp70 (SC66048), and β-actin (SC47778) were purchased from Santa Cruz Biotech, TX, USA. Hsp90 (ADI-SPA-830F) was purchased from Enzo Life Sciences, NY, USA. AC6 (GTX47798) was purchased from GeneTex, Irvine, Calif., USA.

7.3 Cyclic AMP Assay

Confluent cells were treated with VX-809 (10 μM) or DMSO for 16 h before being harvested for assay. Cyclic AMP levels were measured with a direct cAMP Enzyme Immunoassay Kit (Sigma, #CA200) according to the manufacturer's protocol. The results are expressed as pmole/mL.

7.4 Fura-2 Ca2+ Imaging Assay

The cells were loaded with the cell-permeant acetoxymethyl (AM) ester of the calcium indicator fura-2 (fura-2/AM) at 37° C. for 90 min. Measurements were made on a Zeiss inverted microscope equipped with a Sutter Lambda 10-2 controller and filter wheel assembly. A Zeiss FluorArc mercury lamp was used to excite the cells at 340 and 380 nm, and the emission response was measured at 510 nm. Cell fluorescence was measured in response to excitation for 1000 ms at 340 nm and 200 ms at 380 nm once every 4 s. Image acquisition, image analysis, and filter wheel control were performed with IPLab software (see Yanda et al., 2019b).

### Example 8

### ARPKD Pkhd1^{del3-4/del3-4} Mouse Liver and Patient Kidney Cells

During the development of embodiments of the technology described herein, experiments were conducted in a pkhd1^{Pkhd1del3-4/del3-4} deletion model obtained from the Baltimore Polycystic Kidney Disease Center (Garcia-Gonzalez (2007) Genetic interaction studies link autosomal dominant and recessive polycystic kidney disease in a common pathway. Hum. Mol. Genet. 16, 1940-1950). These mice develop biliary proliferation and cysts. Experiments were conducted in untreated mice and pkhd1^{del/4/del4} mice treated with VX-809. Male and female mice (equal numbers) were injected with 30 mg/kg of VX-809 every other day for 30 days beginning at 5 days old the animals were necropsied at 35 days. In addition to this animal model, renal cells were used from a patient 93A-E with ARPKD PKHD1 p. T36M&p. F923C who has two PKHD1 missense mutations. Liver cells from two patients with polycystic liver disease (PLD) were also tested: patient 73: SEC63 c.2091 dupA (Tyr694*) and patient 86:PRKCSH c.367_368celGA obtained from Peter Harris at the Mayo Clinic.

Data collected from these experiments indicated that VX-809 rescued several processes malfunctioning in ARPKD. In particular, the data indicated that VX-809 reduced proliferation, increased CFTR at the basolateral membrane, reduced cAMP, and reduced ER Ca²⁺ release in response to thapsigargin. These response to treatment either individually or in combination reduce cyst growth in liver and thus improve liver, renal, and lung function.

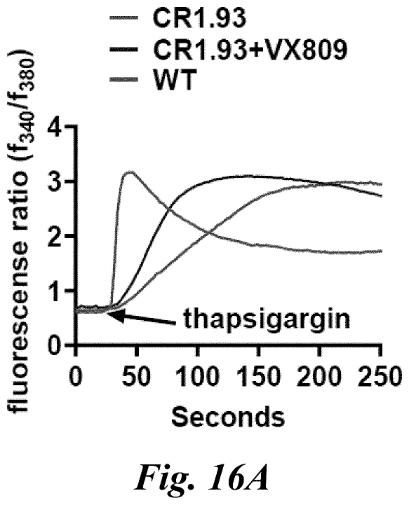

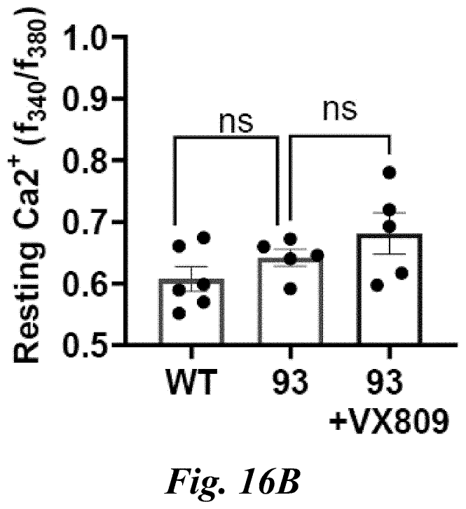

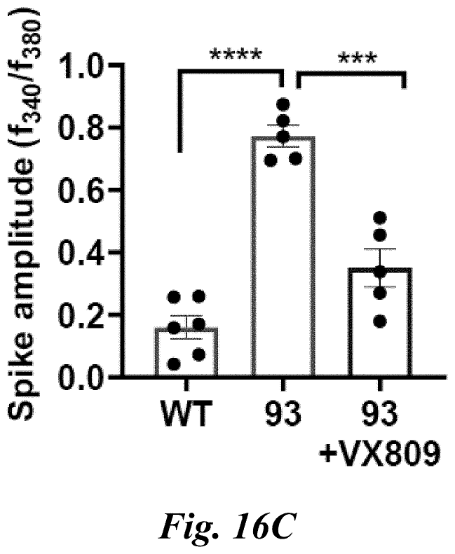

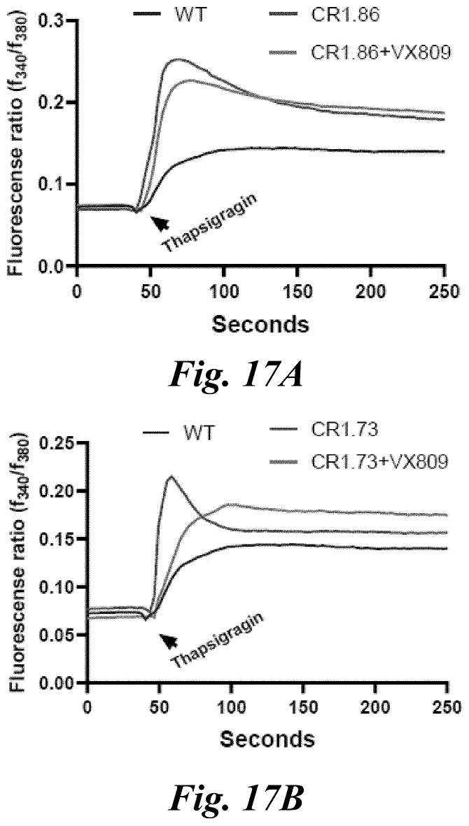

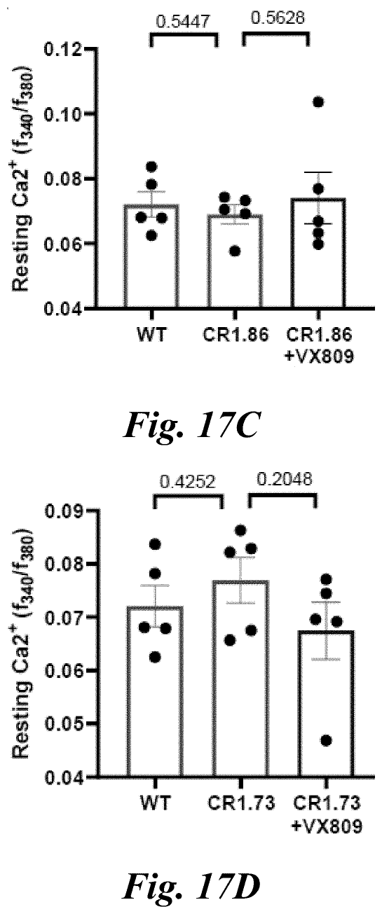

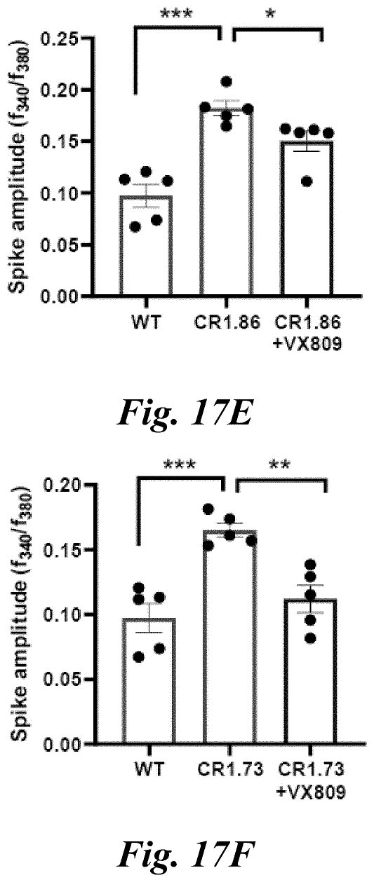

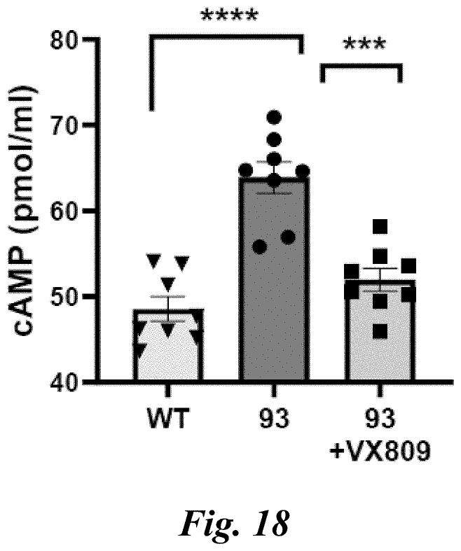

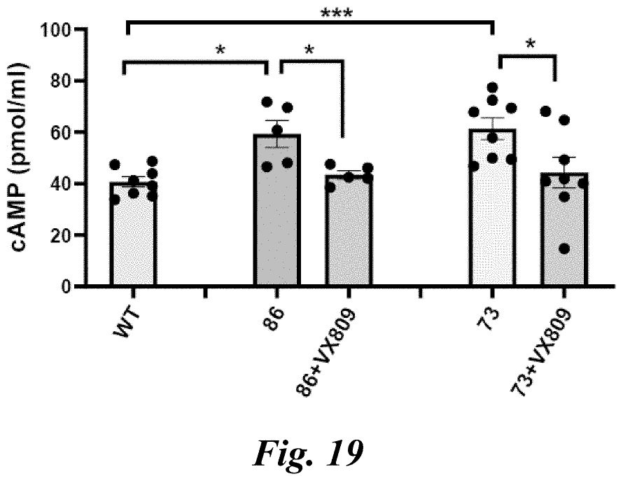

In particular, data were collected indicating that VX-809 downregulates release of Ca²⁺ from the ER: The ER is one of the major repositories for Ca²⁺ in the cell (see, e.g., Burgoyne (2015) Calcium signaling at ER membrane contact sites. Biochim. Biophys. Acta 1853, 2012-2017, incorporated herein by reference). Misregulation of Ca²⁺ signaling is a hallmark of ADPKD (see, e.g., Yanda (2019) Role of calcium in adult onset polycystic kidney disease. Cell. Signal. 53, 140-150, incorporated herein by reference). Experiments were conducted during the development of embodiments of the technology described herein to measure ER Ca²⁺ release by treating cells with thapsigargin, a specific inhibitor of the ER Ca²⁺-ATPase that allows Ca²⁺ to leak from the ER through independent Ca²⁺-permeable pathways (see, e.g., Jackson (1988) A novel tumour promoter, thapsigargin, transiently increases cytoplasmic free Ca2+ without generation of inositol phosphates in NG115-401L neuronal cells. Biochem. J. 253, 81-86; Thastrup (1990) Thapsigargin, a tumor promoter, discharges intracellular Ca2+ stores by specific inhibition of the endoplasmic reticulum Ca2(+)-ATPase. Proc. Natl. Acad. Sci. U.S.A 87, 2466-2470; Thastrup (1989) Thapsigargin, a novel molecular probe for studying intracellular calcium release and storage. Agents Actions 27, 17-23, each of which is incorporated herein by reference). Data collected during these experiments indicated that the magnitude of the thapsigargin-induced increase in intracellular Ca²⁺ was greater in Pkhd1^{del4/del4} cholangiocytes (FIG. 16A and FIG. 16B). This result is consistent with previous data showing that Ca²⁺signaling is elevated in PC1-knockout cells (see, e.g., Li (2009) Polycystin-1 interacts with inositol 1,4,5-trisphosphate receptor to modulate intracellular Ca2+ signaling with implications for polycystic kidney disease. J. Biol. Chem. 284, 36431-36441, incorporated herein by reference). Importantly, VX-809 dramatically reduced the thapsigargin-induced ER Ca²⁺ release to values similar to those observed in WT cells. Further, data were collected indicating that VX-809 downregulates cAMP levels. As shown in FIGS. 18 and 19, human cells isolated from ARPKD and PLD patients have higher cAMP levels than WT cells and VX-809 reduces cAMP levels.

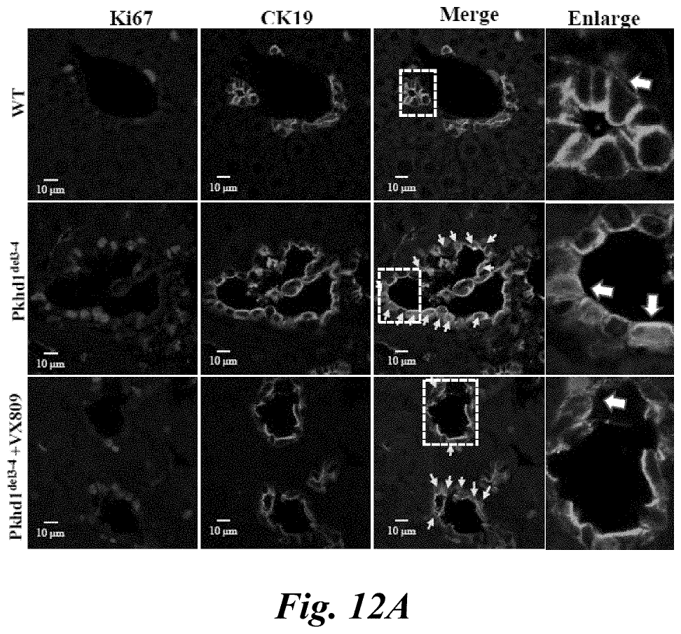

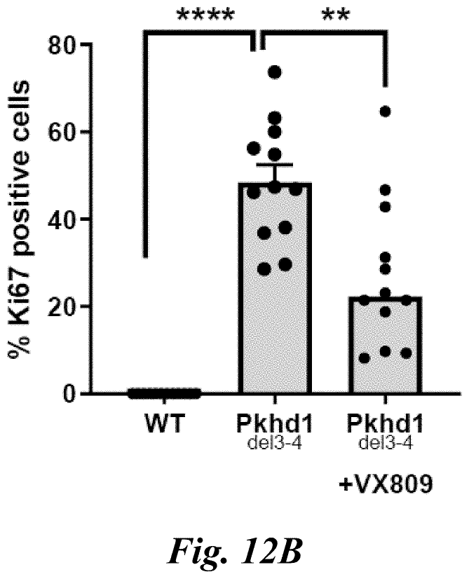

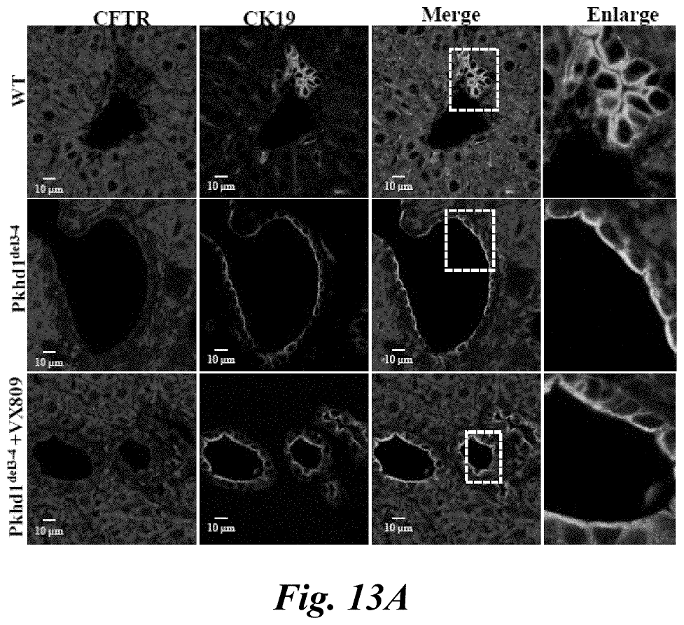

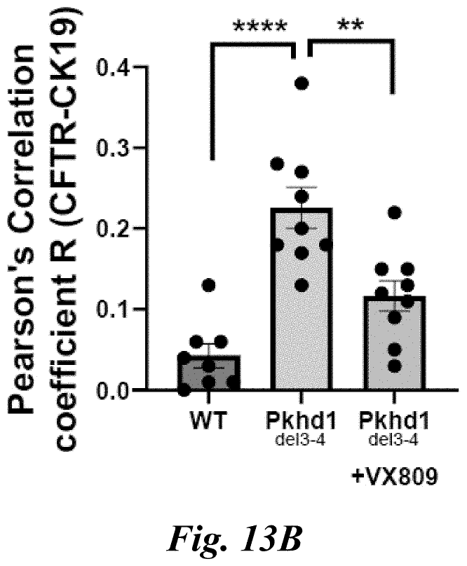

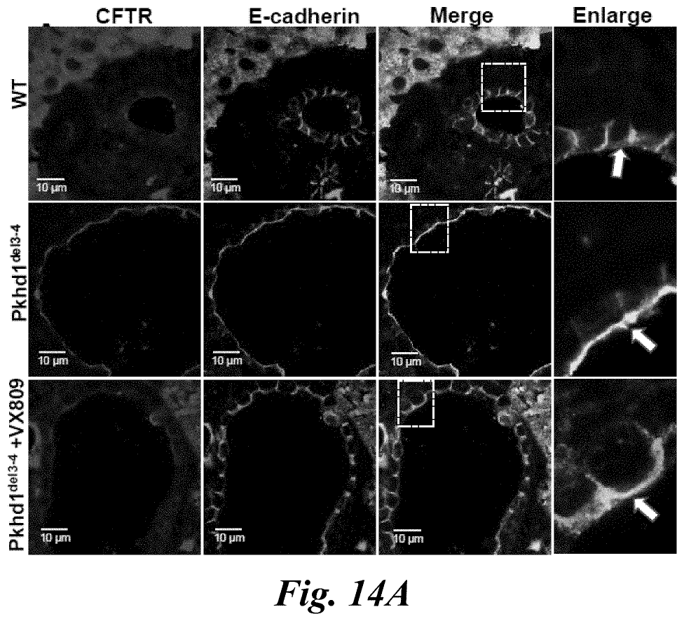

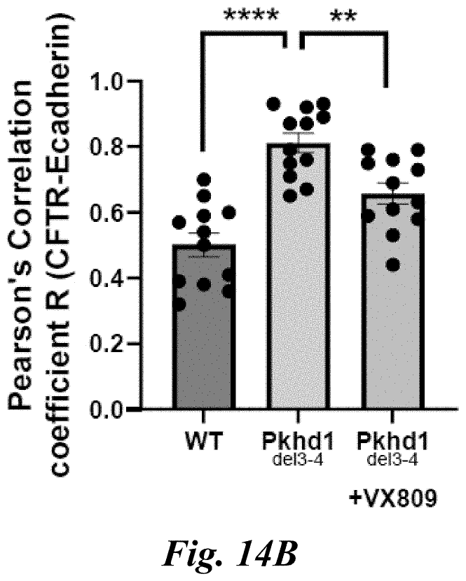

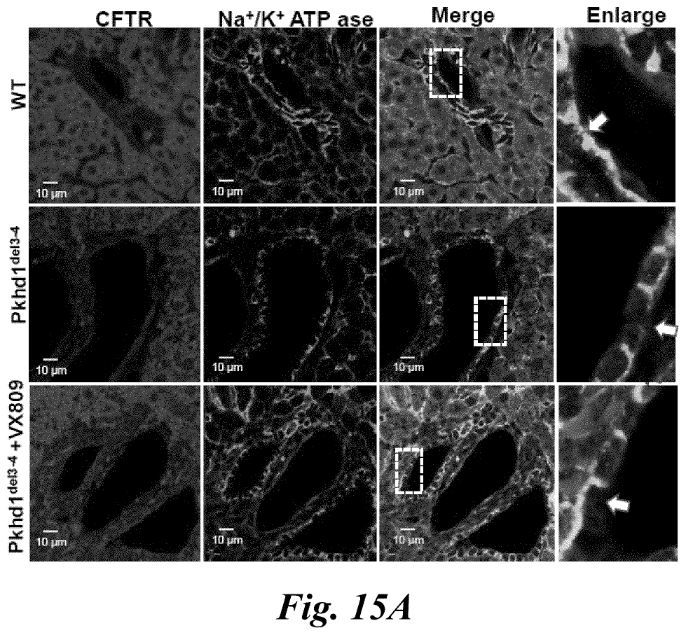

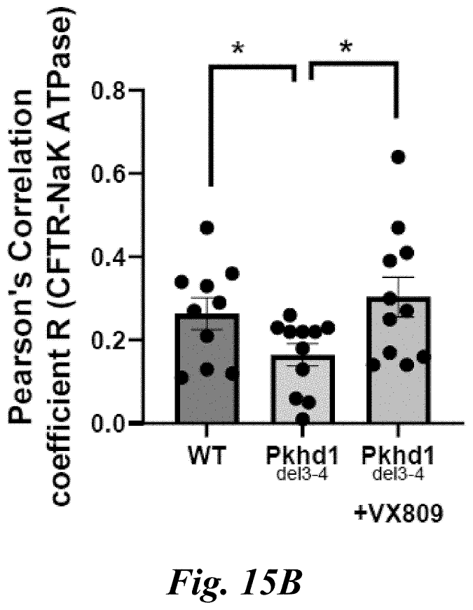

In further experiments, data were collected that indicated that VX-809 reduces proliferation in Pkhd1^{del3-4/del3-4} cholangiocytes of mouse liver. Experiments were conducted during the development of embodiments of the technology described herein to test if CFTR correctors affect proliferation of cholangiocytes. In these experiments, proliferation in the livers of Pkhd1^{del3-4/del3-4} cholangiocytes in bile duct epithelia was measured using confocal microscopy and the proliferation marker Ki67. FIG. 12A and FIG. 12B show that the presence of Ki67 was greatest in Pkhd1^{del3-4/del3-4} cholangiocytes in the bile ducts compared to the normal bile ducts, which had very little staining indicative of low proliferation in the normal cholangiocytes. The CFTR corrector VX-809 reduced proliferation, indicating that VX-809 reduces the growth of cysts in the liver. The dosage of VX-809 injected was lower than the human pediatric dose, which is 500 mg of Lumacaftor per day; the adult dose is 800 mg/day. Further, data collected indicated that loss of FPC function affects the steady the localization of CFTR and favors the localization of CFTR at the apical cell membrane (FIG. 14A and FIG. 14B). Treatment with VX-809 increased the colocalization with the basolateral membrane (FIG. 15A and FIG. 15B). These data indicate that changes in CFTR may contribute significantly to the pathophysiology of ARPKD and that correction toward normal is important therapeutically.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Sweeney, W. E., and Avner, E. D. (2006) Molecular and cellular pathophysiology of autosomal recessive polycystic kidney disease (ARPKD). Cell Tissue Res. 326, 671-685.
Zerres, K., Mucher, G., Becker, J., Steinkamm, C., Rudnik-Schoneborn, S., Heikkila, P., Rapola, J., Salonen, R., Germino, G. G., Onuchic, L., Somlo, S., Avner, E. D., Harman, L. A., Stockwin, J. M., and Guay-Woodford, L. M. (1998) Prenatal diagnosis of autosomal recessive polycystic kidney disease (ARPKD): molecular genetics, clinical experience, and fetal morphology. Am. J. Med. Genet. 76, 137-144.
Roy, S., Dillon, M. J., Trompeter, R. S., and Barratt, T. M. (1997) Autosomal recessive polycystic kidney disease: long-term outcome of neonatal survivors. Pediatr. Nephrol. 11, 302-306.
Hartung, E. A., and Guay-Woodford, L. M. (2014) Autosomal recessive polycystic kidney disease: a hepatorenal fibrocystic disorder with pleiotropic effects. Pediatrics 134, e833-e845.
Onuchic, L. F., Furu, L., Nagasawa, Y., Hou, X., Eggermann, T., Ren, Z., Bergmann, C., Senderek, J., Esquivel, E., Zeltner, R., Rudnik-Schöneborn, S., Mrug, M., Sweeney, W., Avner, E. D., Zerres, K., Guay-Woodford, L. M., Somlo, S., and Germino, G. G. (2002) PKHD1, the polycystic kidney and hepatic disease 1 gene, encodes a novel large protein containing multiple immunoglobulin-like plexin-transcription-factor domains and parallel beta-helix 1 repeats. Am. J. Hum. Genet. 70, 1305-1317.
Harris, P. C., and Rossetti, S. (2004) Molecular genetics of autosomal recessive polycystic kidney disease. Mol. Genet. Metab 81, 75-85.
Menezes, L. F., Cai, Y., Nagasawa, Y., Silva, A. M., Watkins, M. L., Da Silva, A. M., Somlo, S., Guay-Woodford, L. M., Germino, G. G., and Onuchic, L. F. (2004) Polyductin, the PKHD1 gene product, comprises isoforms expressed in plasma membrane, primary cilium, and cytoplasm. Kidney Int. 66, 1345-1355.
Kim, I., Fu, Y., Hui, K., Moeckel, G., Mai, W., Li, C., Liang, D., Zhao, P., Ma, J., Chen, X.-Z., George, A. L., Jr., Coffey, R. J., Feng, Z.-P., and Wu, G. (2008) Fibrocystin/polyductin modulates renal tubular formation by regulating polycystin-2 expression and function. Journal of the American Society of Nephrology: JASN 19, 455-468.
Fuller, C. M., and Benos, D. J. (1992) Cftr! Am. J. Physiol. 263, C267-286.
Garcia-Gonzalez, M. A., Menezes, L. F., Piontek, K. B., Kaimori, J., Huso, D. L., Watnick, T., Onuchic, L. F., Guay-Woodford, L. M., and Germino, G. G. (2007) Genetic interaction studies link autosomal dominant and recessive polycystic kidney disease in a common pathway. Hum. Mol. Genet. 16, 1940-1950.
Paku, S., Dezsö, K., Kopper, L., and Nagy, P. (2005) Immunohistochemical analysis of cytokeratin 7 expression in resting and proliferating biliary structures of rat liver. Hepatology 42, 863-870.
Yanda, M. K., Cha, B., Cebotaru, C. V., and Cebotaru, L. (2019a) Pharmacological reversal of renal cysts from secretion to absorption suggests a potential therapeutic strategy for managing autosomal dominant polycystic kidney disease. J. Biol. Chem. 294, 17090-17104.
Yanda, M. K., Liu, Q., and Cebotaru, L. (2018) A potential strategy for reducing cysts in autosomal dominant polycystic kidney disease with a CFTR corrector. J. Biol. Chem. 293, 11513-11526.
Burgoyne, T., Patel, S., and Eden, E. R. (2015) Calcium signaling at ER membrane contact sites. Biochim. Biophys. Acta 1853, 2012-2017.
Yanda, M. K., Liu, Q., Cebotaru, V., Guggino, W. B., and Cebotaru, L. (2019b) Role of calcium in adult onset polycystic kidney disease. Cell. Signal. 53, 140-150.
Jackson, T. R., Patterson, S., Thastrup, O., and Hanley, M. (1988) A novel tumour promoter, thapsigargin, transiently increases cytoplasmic free Ca2+ without generation of inositol phosphates in NG115-401L neuronal cells. Biochem. J. 253, 81-86.
Thastrup, O., Cullen, P. J., Drobak, B. K., Hanley, M. R., and Dawson, A. P. (1990) Thapsigargin, a tumor promoter, discharges intracellular Ca2+ stores by specific inhibition of the endoplasmic reticulum Ca2(+)-ATPase. Proc. Natl. Acad. Sci. U.S.A 87, 2466-2470.
Thastrup, O., Dawson, A. P., Scharff, O., Foder, B., Cullen, P. J., Drobak, B. K., Bjerrum, P. J., Christensen, S. B., and Hanley, M. R. (1989) Thapsigargin, a novel molecular probe for studying intracellular calcium release and storage. Agents Actions 27, 17-23.
Li, Y., Santoso, N. G., Yu, S., Woodward, O. M., Qian, F., and Guggino, W. B. (2009) Polycystin-1 interacts with inositol 1,4,5-trisphosphate receptor to modulate intracellular Ca2+ signaling with implications for polycystic kidney disease. J. Biol. Chem. 284, 36431-36441.
Lopes-Pacheco, M., Boinot, C., Sabirzhanova, I., Morales, M. M., Guggino, W. B., and Cebotaru, L. (2015) Combination of Correctors Rescue DeltaF508-CFTR by Reducing Its Association with Hsp40 and Hsp27. J. Biol. Chem. 290, 25636-25645.
Seeger-Nukpezah, T., Proia, D. A., Egleston, B. L., Nikonova, A. S., Kent, T., Cai, K. Q., Hensley, H. H., Ying, W., Chimmanamada, D., Serebriiskii, I. G., and Golemis, E. A. (2013) Inhibiting the HSP90 chaperone slows cyst growth in a mouse model of autosomal dominant polycystic kidney disease. Proc. Natl. Acad. Sci. U.S.A 110, 12786-12791.

Although the foregoing subject matter has been described in some detail by way of illustration and example for purposes of clarity of understanding, it will be understood by those skilled in the art that certain changes and modifications can be practiced within the scope of the appended claims.

1-27. (canceled)

28. A method of treating a cystic kidney disease patient, the method comprising:
identifying a patient having cystic kidney disease: and
administering a cystic fibrosis transmembrane conductance regulator (CFTR) modulator to said patient.

29. The method of claim 28, wherein said patient is a human.

30. The method of claim 28, wherein identifying a patient having cystic kidney disease comprises detecting a kidney and/or a liver cyst in said patient.

31. The method of claim 28, wherein the cystic kidney disease is autosomal recessive polycystic kidney disease (ARPKD).

32. The method of claim 28, wherein identifying a patient having cystic kidney disease comprises detecting a mutation in the PKHD1 gene of said patient.

33. The method of claim 28, wherein identifying a patient having cystic kidney disease comprises detecting a decreased expression or activity of fibrocystin/polyductin (FPC) in said patient.

34. The method of claim 28, wherein identifying a patient having cystic kidney disease comprises detecting an increase of CFTR at the apical cell membrane.

35. The method of claim 28, wherein identifying a patient having cystic kidney disease comprises detecting increased cyclic adenosine monophosphate (cAMP) in said patient.

36. The method of claim 28, wherein identifying a patient having cystic kidney disease comprises detecting misregulation of Ca²⁺ signaling in said patient.

37. The method of claim 28, wherein said CFTR modulator is selected from the group consisting of lumacaftor (VX-809), Corr-4a, VRT-325, C18, C4, C3, VX-770, VX-786, 4-phenylbutyrate (4PBA), VRT-532, N6022, miglustat, sildenafil and analogs thereof, ataluren (PTC124), ouabain, roscovitine, suberoylanilide hydroxamic acid, latonduine and analogs thereof, SAHA, FDL169, tezacaftor (VX-661), VX-659, and VX-445.

38. The method of claim 28, wherein said CFTR modulator is VX-809 or FL169.

39. The method of claim 28, further comprising detecting an increase in CFTR at the basolateral membrane and/or cilia after administering the CFTR corrector to said patient.

40. The method of claim 28, further comprising detecting an increase in FPC after administering the CFTR corrector to said patient.

41. The method of claim 28, further comprising detecting a decrease in cAMP after administering the CFTR corrector to said patient.

42. The method of claim 28, further comprising detecting a decrease in the expression or activity of Hsp70, Hsp90, and Aha1 after administering the CFTR corrector to said patient.

43. The method of claim 28, further comprising detecting a reduction in kidney cyst size or number in said patient after administering the CFTR corrector to said patient.

44. The method of claim 28, further comprising administering a stabilizer to said patient after administering the CFTR corrector to said patient.

45. The method of claim 44, wherein said stabilizer is cavosonstat.

46. The method of claim 28, further comprising detecting an increase in correctly processed and trafficked CFTR in said patient.

47. The method of claim 45, further comprising detecting an increase in correctly processed and trafficked CFTR in said patient.
